(19) United States
(12) Patent Application Publication
(10) Pub. No.: US 2026/0199606 A1
(43) Pub. Date: Jul. 16, 2026

(54) NEEDLE SHIELD REMOVER

(71) Applicant: Owen Mumford Limited, Woodstock, Oxfordshire (GB)

(72) Inventors: Abiodun FALODI, Woodstock, Oxfordshire (GB); Jack CALVERT, Woodstock, Oxfordshire (GB); Matthew John DOBSON, Woodstock, Oxfordshire (GB)

(21) Appl. No.: 19/132,326

(22) PCT Filed: Dec. 13, 2022
(86) PCT No.: PCT/EP2022/085506

§ 371 Date: May 22, 2025

(30) Foreign Application Priority Data
Dec. 13, 2021 (GB) 2117955.1

(51) Int. Cl. A61M 5/32

(52) U.S. Cl. CPC A61M 5/3204; A61M 2207/00

(57) ABSTRACT

The present invention relates to a needle shield remover for an injection device including a body, a proximal gripping member extending inwardly from the body to engage a needle shield; and a distal gripping member extending from the body and arranged to engage a cap of the injection device.

### TECHNICAL FIELD

The present disclosure relates to a needle shield remover. In particular, it relates to a needle shield remover, a cap for an invention device and an injection device.

### BACKGROUND

Injection devices including a syringe, such as auto-injectors, are commonly provided with at least one needle shield to protect the syringe needle from damage and accidental contact and to maintain sterility of the injection needle. The needle shield may be a soft needle shield that is formed of a flexible material or a rigid needle shield that is formed of a rigid, inflexible material which provide greater mechanical protection to the injection needle. Conventional injection devices may also include a removable cap covering the needle shields to provide mechanical protection for the needle shields and to facilitate removal of the needle shields before an injection may be performed.

### SUMMARY OF THE INVENTION

Aspects and embodiments of the invention provide a needle shield remover, a cap for an invention device and an injection device as claimed in the appended claims.

According to an aspect of the present invention there is provided a needle shield remover for an injection device including a body, a proximal gripping member extending inwardly from the body to engage a needle shield; and a distal gripping member extending from the body and arranged to engage a cap of the injection device. This retains the needle shield within the cap of an injection device. Therefore, when the cap of the injection device is removed from the injection device the needle shield of a syringe contained within the injection device and engaged with the needle shield remover is removed from the syringe. The needle shield is retained by the needle shield remover until removed by a user.

Optionally, the body comprises a cylindrical wall arranged to define a passageway to receive a needle shield.

The body may be formed from a sheet of metal and the proximal and/or distal gripping members are formed by stamping the sheet metal. Preferably the proximal and/or distal gripping members are integral to the body and formed as elongate tongues, optionally the gripping members are resilient tongues. Preferably they extend at a predetermined angle to the body of the needle shield remover.

Optionally, the proximal gripping member extends distally thereby to retain the needle shield within the passageway more effectively when an axial force is applied to the needle shield remover by removing the cap.

Optionally, the distal gripping member extends proximally to increase the resistance to an axial force applied to the distal gripping members when the cap is removed from the injection device and improve retention of the needle shield remover within the cap of the injection device.

According to another aspect of the present invention there is provided a cap for an injection device comprising: an end wall; a wall extending proximally from the end wall; and a needle shield remover as described above.

The wall extending proximally from the end wall may be an inner wall and the cap further comprises an outer wall extending proximally from the end wall. This allows the inner wall to be appropriately sized to receive a needle shield remover and the outer wall to be configured to be gripped by a user.

Optionally, the needle shield remover is received within the inner wall. When the needle shield remover is received within the inner wall preferably the distal gripping member extends externally from the body to engage with the inner wall.

Optionally, the inner wall is received within the passageway of the needle shield remover. When the inner wall is received within the passageway of the needle shield remover preferably the distal gripping member extends internally from the body to engage with the inner wall.

Preferably the inner wall further comprises a recess and/or aperture to receive a distal member of the needle shield remover. The engagement between the distal gripping member and recess and/or aperture increases the force required to disengage the gripping member and recess and therefore increases the force required to disengage the needle shield remover and cap.

According to another aspect of the present invention there is provided an injection device including the cap or needle shield remover as described above.

### BRIEF DESCRIPTION OF THE DRAWINGS

One or more embodiments of the invention will now be described, by way of example only, with reference to the accompanying drawings, in which:

FIG. 1 illustrates a needle shield remover according to the present invention;

FIG. 2 shows a perspective view of the cap according to the present invention;

FIG. 3 illustrates the needle shield remover according to the present invention being brought into engagement with the cap;

FIG. 4 illustrates the needle shield remover and cap in engagement; and

FIG. 5 is a cross-section through the needle shield remover and cap in engagement.

### DETAILED DESCRIPTION

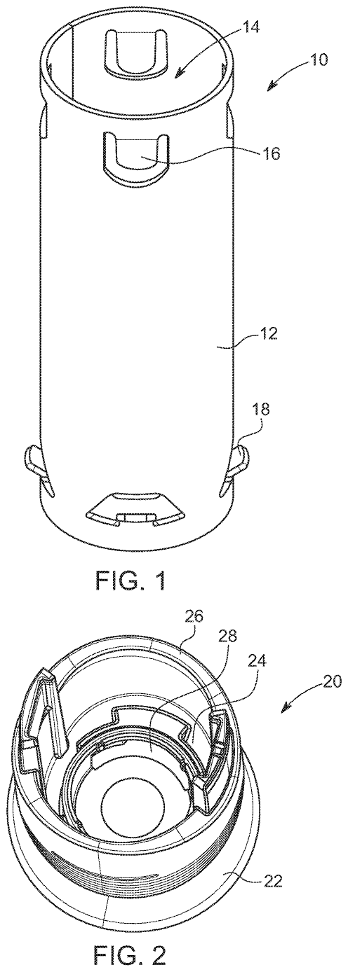

A needle shield remover for use in an injection device according to an embodiment of the present invention is described herein with reference to FIG. 1.

With reference to FIG. 1, the needle shield remover 10 comprises a tubular body 12 that extends along a longitudinal axis. The tubular body 12 of the needle shield remover 10 defines a passageway 14 which is configured to receive a needle shield (not shown). The body 12 further comprises at least two sets of gripping members, proximal gripping members 16 and distal gripping members 18.

The proximal gripping members 16 extend inwardly into the passageway of the needle shield remover 10 and are configured to engage the outer surface of a needle shield. Preferably, if two or more proximal gripping members 16 are provided, the proximal gripping members 16 are arranged in an annular form on the inner circumference of the tubular body 12. The gripping members 16 are preferably a predetermined distance from the proximal end of the tubular body 12.

Preferably each proximal gripping member 16 is provided as an elongate member extending into the interior of the passageway at a predetermined angle in relation to a longitudinal axis of the device and towards the distal end of the needle shield remover. Optionally, the elongate members are resilient tongues made integral with the body 12 of the needle shield remover 10.

The distal gripping members 18 extend outwardly away from the passageway of the needle shield remover 10 and are configured to engage a cap of the injection device. Thereby, causing the cap and the needle shield remover 10 to become attached such that axial movement of the cap causes axial movement of the needle shield remover 10. The attachment may be removeable or fixed. Preferably, if two or more distal gripping members 18 are provided, the distal gripping members 18 are arranged in an annular form on the outer circumference of the tubular body 12. The distal gripping members 18 are preferably a predetermined distance from the distal end of the tubular body 12.

Preferably each distal gripping member 18 is provided as an elongate member extending outwardly from the tubular body 12 at a predetermined angle in relation to a longitudinal axis of the device and towards the proximal end of the needle shield remover. Optionally, the elongate members are resilient tongues made integral with the body 12 of the needle shield remover 10.

The end of one or more of the proximal and distal gripping members 16, 18 are provided with a pointed sharp section to grip the needle shield and the cap respectively.

The needle shield remover 10 may, for example, be formed of a sheet metal with one or more of the gripping members being formed through stamping the shape into a sheet metal. The sheet metal can then be rolled and welded to form the tubular shape. The skilled person will understand that any suitable method may be used to form the needle shield remover 10.

The injection device further comprises a cap 20 as illustrated in FIG. 2. The cap 20 is arranged to be removeably attached to the injection device. The cap 20 preferably has a generally tubular shape with an end surface 22 and inner and outer proximally extending tubular walls 24, 26. The end surface 22 preferably has a generally circular configuration, is perpendicular with reference to the longitudinal axis of the injection device. Further, the end surface extends radially outside the outer tubular wall 24.

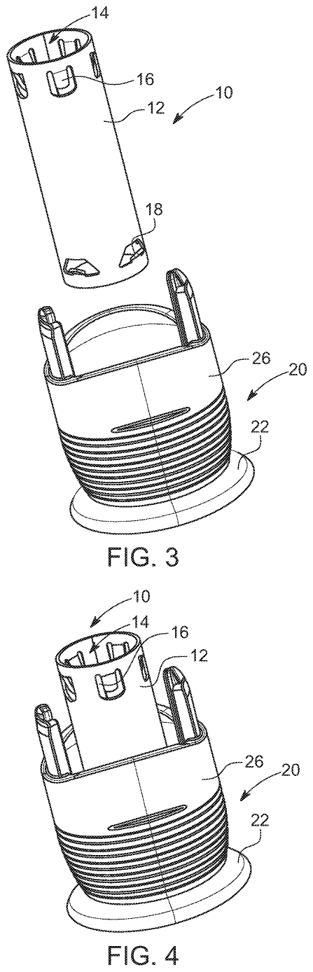

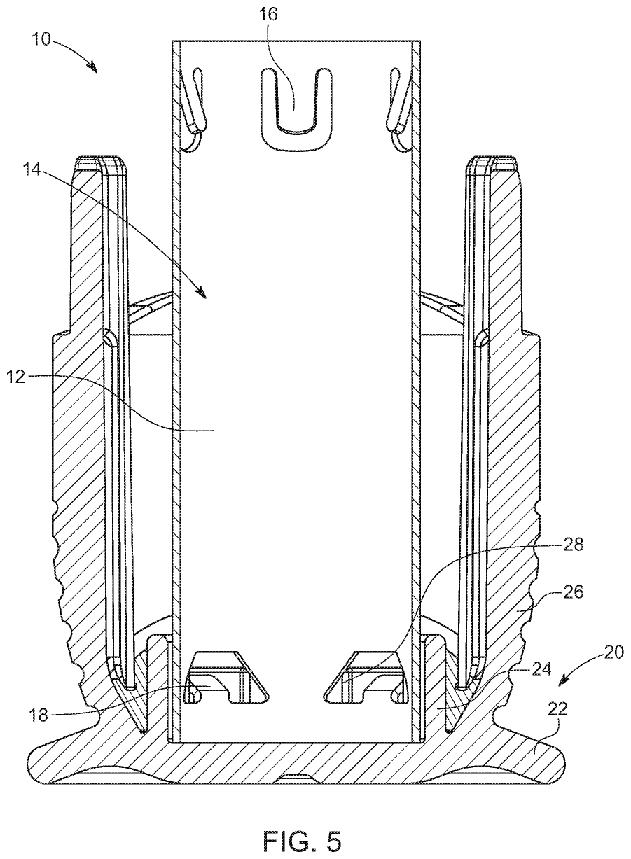

The inner proximally extending tubular wall 24 is sized to receive the tubular body 12 of the needle shield remover 10 within it. The inner tubular wall 24 is preferably provided with one or more recesses 28 on its inner surface, each recess or aperture being configured to receive a corresponding distal gripping member 18 such that the cap and needle shield remover 10 are fixedly connected as illustrated in FIGS. 3 to 5.

Although the needle shield remover 10 has been described as being received within the inner proximally extending tubular wall 24 of the cap the skilled person would understand that, in an alternative formation, the inner proximally extending tubular wall 24 of the cap may be received within the passageway of the needle shield remover 10. In such an instance the one or more recesses may be provided on the outer surface of the inner proximally extending tubular wall 24 and the distal gripping members 18 extend inwardly into the passageway of the tubular body 12 so that they engage with the one or more recesses.

Although the gripping members have been described as being received within recesses; the skilled person will understand that the recesses may, alternatively, be apertures in the inner wall 24. Alternatively, a mixture of recesses and apertures may be used. Optionally, the inner wall 24 may not be provided with any apertures or recesses and the needle shield remover 10 and inner wall 24 are held in engagement through the distal gripping members 18 engaging with the inner or outer surface of the inner wall 24. The skilled person will also understand that the inner wall may be provided with a mixture of apertures and recesses and further that each distal gripping member 18 may not be provided with a corresponding recess/aperture so that some of the distal gripping members 18 are received by a recess/aperture and one or more distal gripping members 18 engage with the inner or outer surface of the inner wall 24.

Although the body has been described as tubular the skilled person would understand that the body may be any suitable shape.

Additionally, although the needle shield remover has been described as being made from metal the skilled person would understand that any suitable material may be used.

Certain terminology is used in the following description for convenience only and is not limiting. The word “distal” refers to the front, or patient, end of the device. The word “proximal” refers to the rear, or syringe user, end of the device. The term “longitudinal”, with or without axis, refers to a direction on an axis through the device in the direction of the longest extension of the device. The term “radial” or “transverse/transversal”, with or without axis, refers to a direction generally perpendicular to the longitudinal direction, e.g. “radially outward” would refer to a direction pointing away from the longitudinal axis.

It should be understood that the detailed description and specific examples are given by way of illustration only, since various changes and modifications within the spirit and scope of the disclosure will become apparent to those skilled in the art from the description.

1. A needle shield remover for an injection device comprising: a body; a proximal gripping member extending inwardly from the body to engage a needle shield; and a distal gripping member extending from the body and arranged to engage a cap of the injection device.

2. A needle shield remover according to claim 1 wherein the body comprises a cylindrical wall arranged to define a passageway to receive a needle shield.

3. A needle shield remover according to claim 2 wherein the elongate member is formed from a sheet of metal and the proximal and/or distal gripping members are formed by stamping the sheet metal.

4. A needle shield remover according to claim 1 wherein the proximal and distal gripping members are elongated.

5. A needle shield remover according to claim 1 wherein the proximal gripping member extends distally.

6. A needle shield remover according to claim 1 wherein the distal gripping member extends proximally.

7. A cap for an injection device comprising: an end wall; a wall extending proximally from the end wall; and a needle shield remover according to claim 1.

8. A cap for an injection device according to claim 7 wherein the wall extending proximally from the end wall is an inner wall and the cap further comprises an outer wall extending proximally from the end wall.

9. A cap for an injection device according to claim 8 wherein the needle shield remover is received within the inner wall.

10. A cap for an injection device according to claim 9 wherein the distal gripping member extends externally from the body to engage with the inner wall.

11. A cap for an injection device according to claim 8 wherein the inner wall is received within the passageway of the needle shield remover.

12. A cap for an injection device according to claim 11 wherein the distal gripping member extends internally from the body to engage with the inner wall.

13. A cap for an injection device according to claim 8 wherein the inner wall further comprises a recess to receive a distal member of the needle shield remover.

14. A cap for an injection device according to claim 8 wherein the inner wall further comprises an aperture configured to receive a distal member of the needle shield remover.

15. An injection device including the cap according to claim 7.
